(19) United States
(12) Patent Application Publication
(10) Pub. No.: US 2024/0390667 A1
(43) Pub. Date: Nov. 28, 2024

(54) ELECTRODE SET AND DEVICE FOR TRANSPARENTIZING BIOLOGICAL TISSUE

(71) Applicant: NATIONAL TSING HUA UNIVERSITY, Hsinchu City (TW)

(72) Inventors: LI-AN CHU, Hsinchu City (TW); ANN-SHYN CHIANG, Hsinchu City (TW); TSU-CHIN CHOU, Hsinchu City (TW); WEI-KUN CHANG, Hsinchu City (TW); YA-HUI LIN, Hsinchu City (TW); PEI-YUN LU, Hsinchu City (TW)

(73) Assignee: NATIONAL TSING HUA UNIVERSITY, Hsinchu City (TW)

(21) Appl. No.: 18/656,989

(22) Filed: May 7, 2024

(30) Foreign Application Priority Data
May 25, 2023 (TW) 112119473

(51) Int. Cl. A61N 1/04; A61N 1/20

(52) U.S. Cl. CPC A61N 1/0476; A61N 1/205

(57) ABSTRACT

An electrode set for making a biological tissue transparent includes a first electrode and a second electrode. The first electrode and the second electrode are configured to provide an electric field for the biological tissue. The first electrode has a carrier metal and a coating metal at least partially coated on the carrier metal.

### FIELD OF THE INVENTION

The present invention relates to an electrode set and a device for transparentizing a biological tissue.

### BACKGROUND OF THE INVENTION

Tissue slice imaging is commonly used in the study of biological tissues (such as animal and/or human experiments). However, the planar images (2D) or composite images (3D) generated by sliced tissues have limitations in analyzing the structural distribution of biological cells or biological tissues. For example, analysis errors or misjudgments may occur due to deformation of sliced tissues caused by slicing methods or slicing instruments, or image processing defects such as misalignment or overlap when combining images. Therefore, it is necessary and important to directly establish complete 3D images for the study of biological tissues.

However, most of the biological tissues are opaque or not light-transparent. Therefore, when establishing a 3D image, opaque tissues will affect the clarity, resolution, or recognizability of the 3D image. For example, thick or dark color tissues will have poor transparency and affect the resolution of the target object. Therefore, making a biological tissue transparent (or transparentizing a biological tissue) is a necessary step for preprocessing biological tissues.

The conventional methods for transparentizing biological tissues can be roughly divided into two types, passive and active. The passive transparentizing methods are cost-effective for only requiring reagents and eliminating the need of additional electronic or mechanical equipment. However, the passive transparentizing methods require more time and have limited transparency effect. Compared to the passive transparentizing methods, the active transparentizing methods can significantly reduce the time required for transparentizing and achieve better results (such as higher light transmittance). Therefore, for a complete tissue processing, the active transparentizing methods and devices have a higher industrial value in practical industrial applications.

In the technology field of active biological tissue transparency, charged substances (such as fats) in a biological tissue are mainly separated through electrophoresis. However, in conventional technologies, if the electrophoresis parameters of the device are not set properly, the biological tissue will overreact and cause the deterioration or deformation of the sample, or the occurrence of precipitates or impurities. Moreover, improper selections of electrophoresis devices or components may deteriorate the device or component itself or generate precipitates, resulting in low efficiency or damage of the biological tissue. The occurrence of precipitates or impurities and/or the devices or components prone to deterioration will increase the costs and waste the consumables. For example, in order to handle the precipitates or impurities, additional thin films or filter membranes are required, and the additional thin films or filter membranes will increase the cost of reagent or consumables. In other example, when the electrophoresis component deteriorates or is damaged, the replacement of the electrophoresis component is necessary. In addition, the setting of the electrical field may be adjusted (for example, using lower current/voltage, or using time-varying electrical fields) to avoid the deterioration of the electrode for electrophoresis or the generation of impurities/precipitates. The adjusted electrical field may increase the operating time required for making the biological tissue transparent.

### SUMMARY OF THE INVENTION

One object of the present invention is to improve the efficiency of transparentizing the biological tissues.

One object of the present invention is to reduce the cost of making the biological tissues transparent.

One object of the present invention is to reduce or avoid the generation of precipitates and/or impurities that may affect the sample during the process of transparentizing the biological tissue.

The present invention provides an electrode set for making a biological tissue transparent. The electrode set includes a first electrode and a second electrode. The first electrode and the second electrode are configured to provide an electric field to the biological tissue. The first electrode has a carrier metal and a coating metal at least partially coated on the carrier metal.

The present invention provides a device for making a biological tissue transparent. The device includes an electrode set, a chamber for accommodating the biological tissue, and a power supply. The electrode set includes a first electrode and a second electrode. The first electrode and the second electrode are configured to provide an electric field to the biological tissue. The first electrode has a carrier metal and a coating metal at least partially coated on the carrier metal. The electrical field is provided in the chamber. The power supply is configured to provide a working current to the electrode set to generate the electrical field.

Regarding the electrode set and device for transparentizing biological tissues mentioned above, since the first electrode has the coating metal coated on the carrier metal, the coating metal will stabilize the first electrode and will not react with the sample or the buffer solution. Such a configuration of the first electrode will ensure that the first electrode has sufficient material stability. In the process of transparentizing the biological tissues, the first electrode which serves as the anode can be prevented from deterioration. Moreover, by using the electrode set of the present invention, means such as fixed electrical field or current can be used due to the stability of the electrode set. Therefore, the efficiency of electrophoresis will be improved, and the deterioration of the electrode set can be prevented.

### BRIEF DESCRIPTION OF THE DRAWINGS

The accompanying drawings are presented to aid in the description of various aspects of the disclosure and are provided solely for illustration of the aspects. To simplify the drawings and highlight the contents to be presented in the drawings, the well-known structures or elements in the drawings may be drawn in a simple schematic manner or presented in an omitted manner. For example, the number of elements may be singular or plural. These drawings are provided only to explain these aspects and not to limit thereof.

FIG. 1 is a schematic diagram of the device for making a biological tissue transparent according to an embodiment of the present invention.

FIG. 2 is a schematic diagram of the device having the electrolyte driver according to an embodiment of the present invention.

FIG. 3 is a schematic diagram of the device having the cooling system according to an embodiment of the present invention.

FIGS. 4A and 4B are schematic sectional views of the first electrode according to embodiments of the present invention.

FIG. 5 is a schematic diagram of the electrode having a mesh structure according to an embodiment of the present invention.

FIGS. 6A and 6B are photographs of the biological tissue after the process of transparentizing according to an embodiment of the present invention.

FIGS. 7A, 7B, and 7C are photographs showing the precipitates and the deteriorated electrode in the chamber after the process of transparentizing by using different electrode sets.

### DETAILED DESCRIPTION OF THE PREFERRED EMBODIMENTS

Even though the terms such as “first”, “second”, and “third” may be used to describe an element, a part, a region, a layer, and/or a portion in the present specification, these elements, parts, regions, layers and/or portions are not limited by such terms. Such terms are used to differentiate an element, a part, a region, a layer, and/or a portion from another element, part, region, layer, and/or portion. Therefore, in the following discussions, a first element, portion, region, or portion may be called a second element, portion, region, layer, or portion, and do not depart from the teaching of the present disclosure. The terms “comprise,” “include”, or “have” used in the present specification are open-ended terms and mean to “include”, but not limit to.

As used herein, the term “coupled to” in the various tenses of the verb “couple” may mean that element A is directly connected to element B or that other elements may be connected between elements A and B (i.e., that element A is indirectly connected with element B).

The terms “approximate” or “essentially” used in the present specification include the value itself and the average values within the acceptable range of deviation of the specific values confirmed by a person having ordinary skill in the current art, considering the specific measurement discussed and the number of errors related to such measurement (that is, the limitation of the measurement system). For example, “about” may mean within one or more standard deviations of the value itself or ±30%, ±20%, ±10%, ±5%. In addition, “about”, “approximate”, or “essentially” used in the present specification may select a more acceptable range of deviation or standard deviation based on optical property, etching property, or other properties. One cannot apply one standard deviation to all properties.

In the present invention, the term of “biological tissue” may refer, for example, to organs, tumors, or other cell aggregates. In the present invention, the term of “biological tissue” not only refers to samples from humans but also animals or plants, such as experimental mice, economic animals, pets, economic crops, or other animals and plants. In the present invention, the term of “biological tissue” is preferably sampling from an organ of the biological subject, such as the brain, kidneys, heart, liver, and other organs of the biological subject, but not limited to.

In the present invention, the term of “transparentize” or “transparentizing” refers to removing at least a portion of components having low transparency from the biological tissue. The components having low transparency may be, for example, proteins, collagen, pigments, fats etc., but not limited to. It should be noted that the “transparentized” product does not necessarily be fully transparent without any scattering, reflection, or other optical phenomena in the product. Instead, for the biological tissues processed by transparentizing, the transparency is improved compared to before transparentizing. In other words, the term of “transparentize” or “transparentizing” of the present invention refers that the scattering and/or attenuation of light transmitted between two points of the biological tissues after the transparentizing treatment is reduced compared to the original biological tissues.

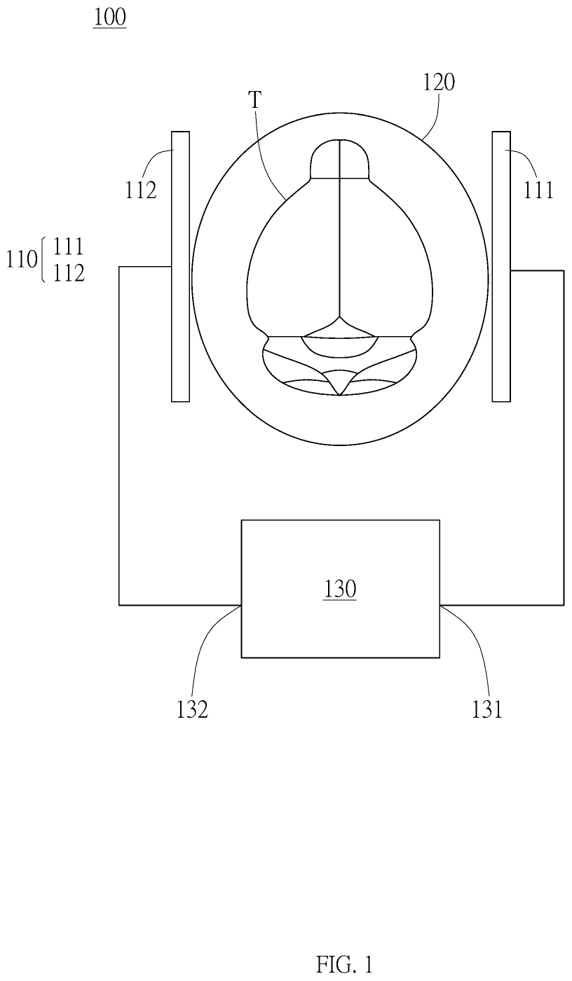

Referring to FIG. 1, FIG. 1 illustrates the device 100 for transparentizing the biological tissue T. The device 100 includes the electrode set 110, the chamber 120 configured to accommodate the biological tissue T, and the power supply 130 configured to provide the working current I to generate the electrical field E. The electrical field E is provided in the chamber 120.

The electrode set 110 includes the first electrode 111 and the second electrode 112. The first electrode 111 is coupled to the positive end 131 of the power supply 130, and the second electrode 112 is coupled to the negative end 132 of the power supply 130. Each of the first electrode 111 and the second electrode 112 is arranged on a side surface of the chamber 120, and the first electrode 111 faces the second electrode 112. More specifically, the chamber 120 can be a cylindrical or cubic shaped chamber. Taking the cylindrical shaped chamber as an example, the biological tissue T is preferably disposed at the center position of the cylindrical shaped chamber, and the first electrode 111 and the second electrode 112 are disposed on the cylindrical wall at two opposite sides of the biological tissue T and face each other. In the process of transparentizing, the chamber 120 can be filled with electrolyte. Then, an electrical signal is provided to the electrode set 110 by the power supply 130 to generate the electrical field E within the chamber 120. During the process of transparentizing, the opaque substances in the biological tissue T can be removed through migration means such as electrophoresis.

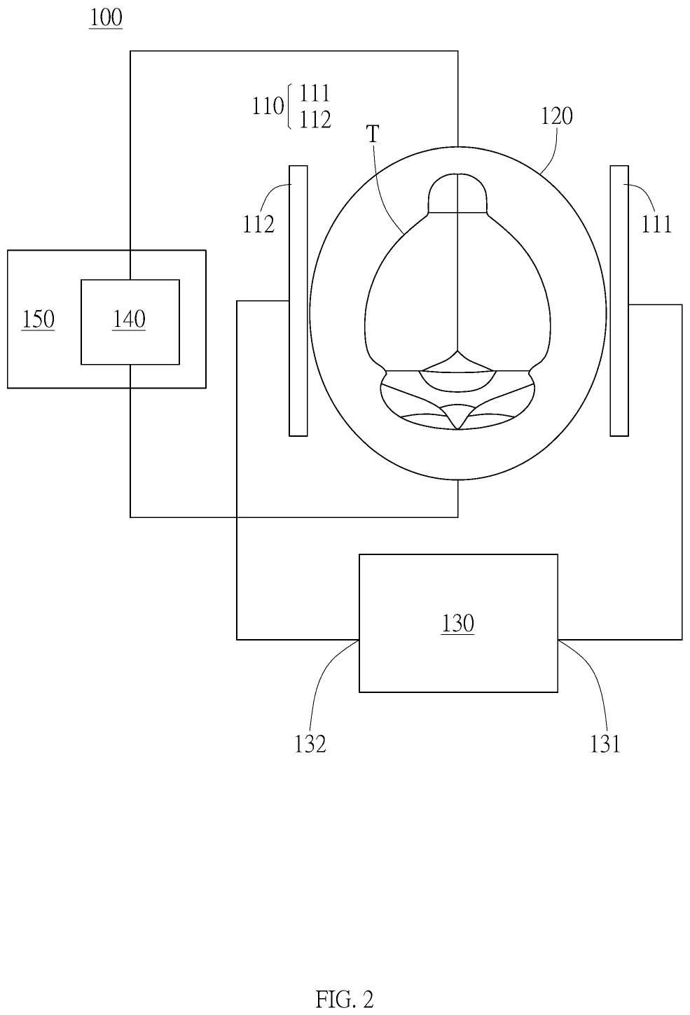

In an embodiment, referring to FIG. 2, the device 100 may further include the electrolyte driver 140 and/or the cooling system 150. More specifically, the electrolyte driver 140 can circulate or replace the electrolyte inside the chamber 120 to effectively address the problem caused by the electrolyte deterioration during the process of transparentizing. However, the purpose of disposing the electrolyte driver 140 is not limited to the embodiment. In addition, as shown in FIG. 2, the cooling system 150 can be installed outside the electrolyte driver 140. For example, the cooling system 150 can be a water bath, air-cooler, water-cooler, or other cooling means. The electrolyte in the chamber 120 can be maintained at a desired temperature (e.g. 18-25° C.) by exchanging heat to or cooling the electrolyte in the electrolyte driver 140 through the cooling system 150. The cooled electrolyte will avoid damage or impact on the biological tissue T caused by high electrolyte temperature during the process of transparentizing. It should be noted that the desired temperature of the electrolyte can be any temperature, and the present invention is not limited to the target temperature of the cooling system 150.

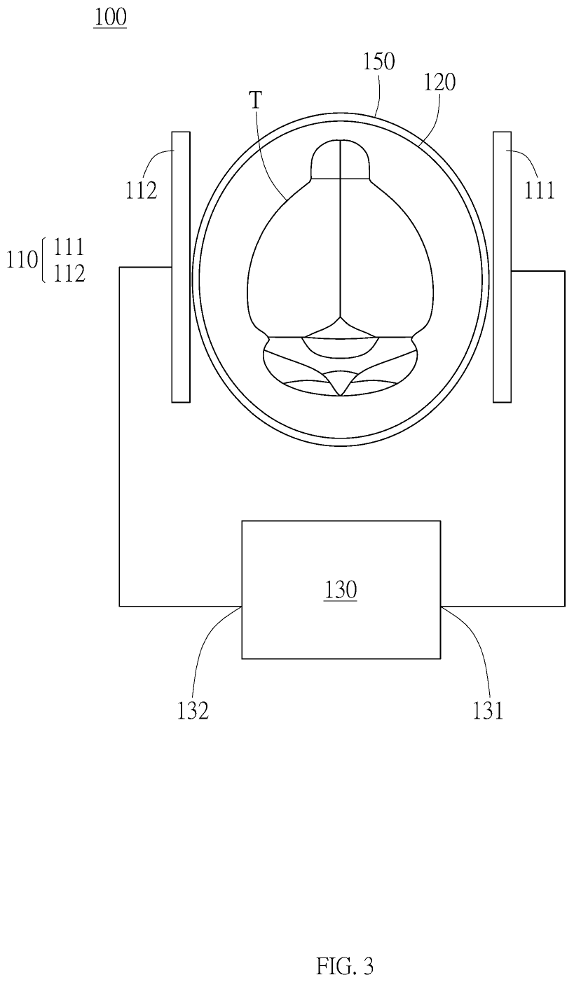

In an embodiment, referring to FIG. 3, the cooling system 150 can be directly integrated with the chamber 120. For example, the chamber 120 can be fabricated by materials with good thermal conductivity. Therefore, the electrolyte inside the chamber 120 can be directly cooled by thermal conduction from the chamber 120 to the cooling system 150, which is integrated with the chamber 120. In the embodiment, the cooling system 150 can be means such as air-cooler, water-cooler, or thermoelectric (TE) cooler. By directly combining the cooling system 150 with the chamber 120, the size of the device 100 can be effectively reduced, and the electrolyte inside the chamber 120 can be cooled to avoid the deterioration of the biological tissue T caused by thermal reactions. It should be noted that although FIG. 3 is not illustrated with the electrolyte driver 140, in the embodiment, the electrolyte circulation or replacement in the chamber 120 can be carried out by the electrolyte driver 140.

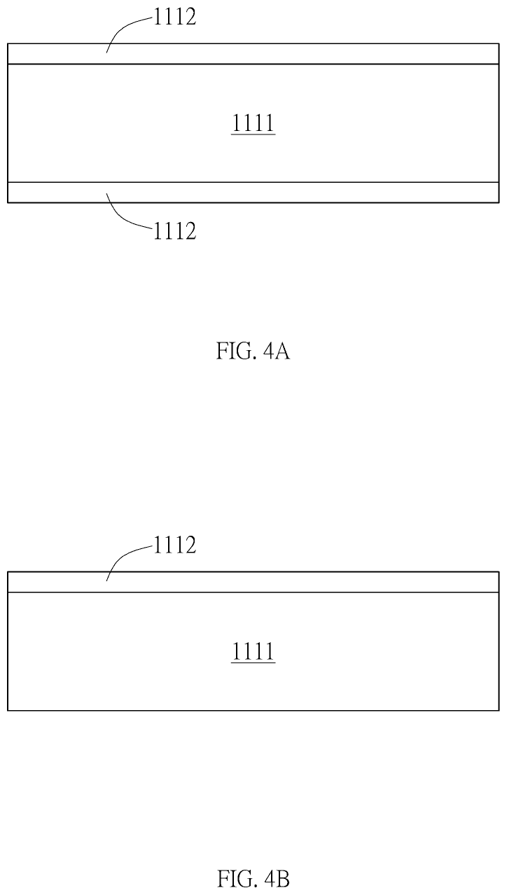

In an embodiment, referring to FIGS. 4A and 4B, the first electrode 111 has the carrier metal 1111 and the coating metal 1112 at least partially coated on the carrier metal 1111. More specifically, the carrier metal 1111 serves as the main structure of the first electrode 111 to provide the mechanical strength. Preferably, the carrier metal 1111 is made by titanium metal. The carrier metal 1111 made by titanium metal can achieve the benefits such as improving current density, corrosion resistance, and/or being less prone to deterioration during electrophoresis, but not limited to. In addition, the coating metal 1112 at least partially covers the carrier metal 1111. More specifically, as shown in FIG. 4A, the coating metal 1112 can completely cover the carrier metal 1111. Alternatively, as shown in FIG. 4B, the coating metal 1112 only covers a portion of the carrier metal 1111. For example, the coating metal 1112 only covers a side of the carrier metal 1111 facing the biological tissue T, and the other side of the carrier metal 1111 opposite to the biological tissue T is not covered by the coating metal 1112. Only covering a portion of the carrier metal 1111 can reduce the usage of the coating metal 1112, and effectively reduce the costs when the coating metal 1112 is a precious metal. However, the purpose and the formation of the coating metal 1112 are not limited to the above examples. For example, the terminal portion of the carrier metal 1111 connected to the power supply 130 may not be covered by the coating metal 1112 or may be processed by a conventional surface treatment such as gold plating. The coating metal 1112 is selected from, for example but not limited to, metals such as ruthenium, iridium, platinum, or their oxides, and iridium or iridium oxide is preferred. The coating metal 1112 can be coated on the carrier metal 1111, which is activated, to enhance the electrochemical reaction characteristics of the first electrode 111.

In a preferred embodiment, the first electrode 111 is a titanium based mixed noble metal oxide coated titanium anode or a dimensionally stable anode. More specifically, the dimensionally stable anode is made by titanium metal or a metal containing titanium (the carrier metal 1111) covered with iridium, ruthenium, or their oxides (the coating metal 1112). The characteristic of dimensionally stable anodes lies in the chemical stability and the electrochemical stability, which enable the first electrode 111 to have a longer service life and a better stability in a high current density and/or a high electrical field working environments. Moreover, the cost of the first electrode 111 made by dimensionally stable anode is relatively lower than conventional electrodes (such as platinum electrodes). From mechanical aspect, the carrier metal 1111 made by titanium metal or metals containing titanium also has advantages such as light weight and/or strong mechanical strength.

In an embodiment, the size of the first electrode 111 is, for example, 30 mm×30 mm×0.1 mm. It should be noted that the present invention is not limited to the size of the first electrode 111. The size of the first electrode 111 can be adjusted according to the size of the chamber 120, the biological tissue T, and/or the current/electrical field required for transparentizing. For example, when transparentizing a larger biological tissue T (for example, the viscera or brain of a pig), the size of the chamber 120, the first electrode 111 and/or the second electrode 112 can be selected according to the volume of the biological tissue T.

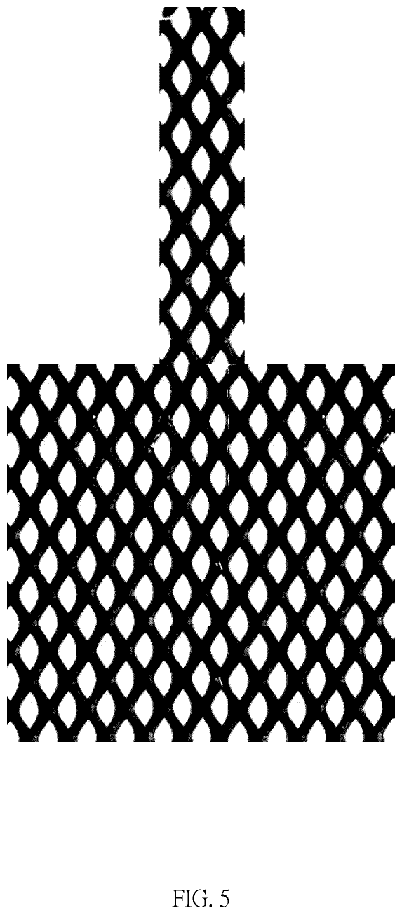

In an embodiment, referring to FIG. 5, the first electrode 111 can be formed as a mesh. More specifically, the carrier metal 1111 can be formed/constructed as a mesh/grid structure. In addition, the present invention is not limited to the shape of the mesh. It should be noted that, although FIG. 5 illustrates diamond shaped mesh, people skilled in the art may know that the mesh can be other shapes, such as triangles, quadrilaterals, or polygons. Through the mesh/grid structure mentioned above, the reaction density and the electrophoresis efficiency of the first electrode 111 can be improved. More specifically, the mesh structure will increase the surface area that can withstand current of the first electrode 111. Accordingly, the mesh structure will make the first electrode 111 to maintain stability at a higher current density during electrophoresis. Thereby, the efficiency of transparentizing can be improved. It should be noted that the mesh structure is not limited to the first electrode 111. For example, the second electrode 112 may also be formed in the same or different mesh structure as the first electrode 111. Regarding the mechanical aspect, the flexibility of disposing the second electrode 112 and/or the first electrode 111 can be improved. Regarding the electrical aspect, the current density of the second electrode 112 and/or the first electrode 111 can be matched to improve the uniformity of the electrical field for electrophoresis.

In an embodiment, the second electrode 112 is used as the cathode. Therefore, the material of the second electrode 112 is selected from conductors having a working voltage (electrochemical voltage) lower than the first electrode 111. It should be noted that the second electrode 112 is not limited to metals, and can be non-metallic materials with conductive properties such as carbon. The second electrode 112 is preferably a nickel metal, which can provide a stable hydrogen evolution reaction and a lower cost to achieve a better transparency effect.

The power supply 130 can be a current or voltage supply. In an embodiment, the power supply 130 is a DC power supply and is preferably a current supply that provides a constant current or a voltage supply that provides a constant voltage. More specifically, when a current supply provides a constant current during the process of transparentizing, the voltage between the first electrode 111 and the second electrode 112 will be varied in a range of 50V to 100V due to the change in impedance of the electrolyte or electrode set during the process. On the other hand, a voltage supply can be used to provide a constant voltage to the electrode set 110 to generate the working current I. The working current I may become variable due to the impedance change of the electrolyte or electrode set. By providing a DC power, the reaction direction of the transparentizing process can be fixed, thereby improving the efficiency of the process. The first electrode 111 and the second electrode 112 of the electrode set 110 disclosed in the present invention will avoid the damage or the deterioration caused by the fixed reaction direction of the process of transparentizing. Therefore, compared to the conventional electrode set, a DC power supply is available without damaging or deteriorating the electrode set 110.

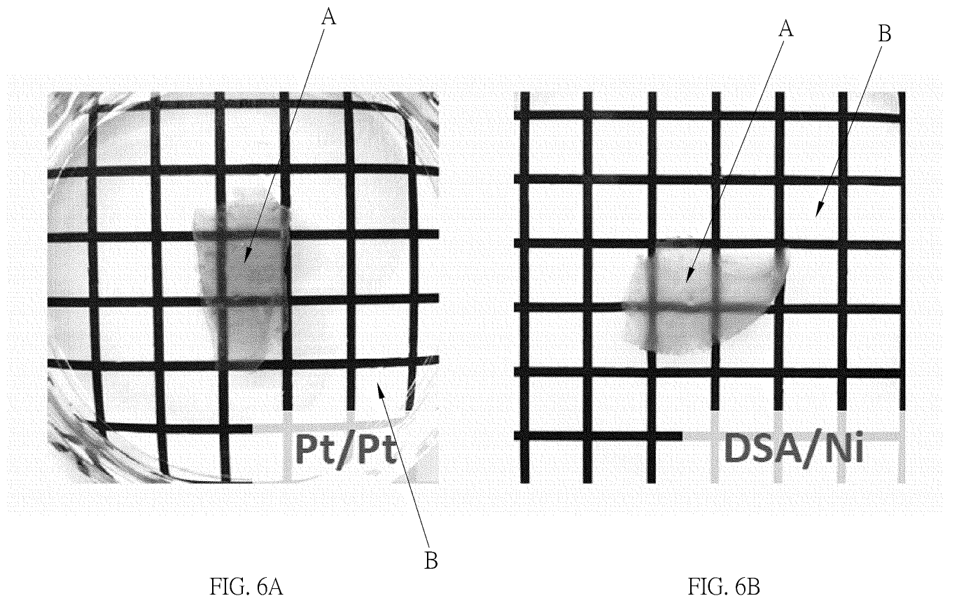

Based on the architecture of the electrode set 110, comparisons between the electrode set 110 of the present invention and the conventional platinum electrode set under the same experimental architecture are provided. In the electrode set 110, a dimensionally stable anode is used as the first electrode 111, and a nickel metal is used as the second electrode 112. The comparison results are shown in FIG. 6A, FIG. 6B, and Table 1. Referring to FIG. 6A and Table 1, the platinum electrode set takes 343 hours to achieve only 58.6% transparency. Referring to FIG. 6B and Table 1, the electrode set 110, which uses the dimensionally stable anode as the first electrode 111 and the nickel metal as the second electrode 112, will achieve 69.2% transparency within 98 hours. It should be noted that the transparency is compared to the grayscale values of the white blocks by using a grayscale meter. More specifically, the transparency is derived from the ratio of the grayscale values of the white blocks covered by the tissue (point A) to the grayscale values of the white blocks not covered by the tissue (point B). Accordingly, the better transparency result is that the grayscale value of the point A is similar or same as the grayscale value of the point B. Therefore, the ratio of transparency close to 1 with a shorter reaction time will provide a better efficiency and a better effect of transparency.

TABLE 1
Comparison of the Electrode Sets

| | Grayscale | Grayscale | | time |
|---|---|---|---|---|
| electrode set | (point A) | (point B) | Transparency | (hr) |
| Conventional | 138.85 | 236.79 | 58.6% | 343 |
| The present invention | 170.76 | 246.73 | 69.2% | 98 |

It should be noted that the transparency parameters and time consumption shown in Table 1 are only intended to explain the level of optimization between the present invention and the conventional electrode set. Table 1 does not intend to limit the reaction time or the transparency effect of the present invention. The comparison for the transparency effect can be analyzed by other image processing means. In other words, the comparison is not necessarily limited to the ratio between the grayscale value of the point A and the grayscale value of the point B. For example, grayscale values can be normalized through image processing to eliminate errors caused by lighting and angles during image capture. In addition, the required time for transparency may also vary due to the size or setting position of the electrode set 110, the size of the chamber 120, or the size or type of the sample. The electrode set and the device of the present invention are not limited by the reaction time of the above examples.

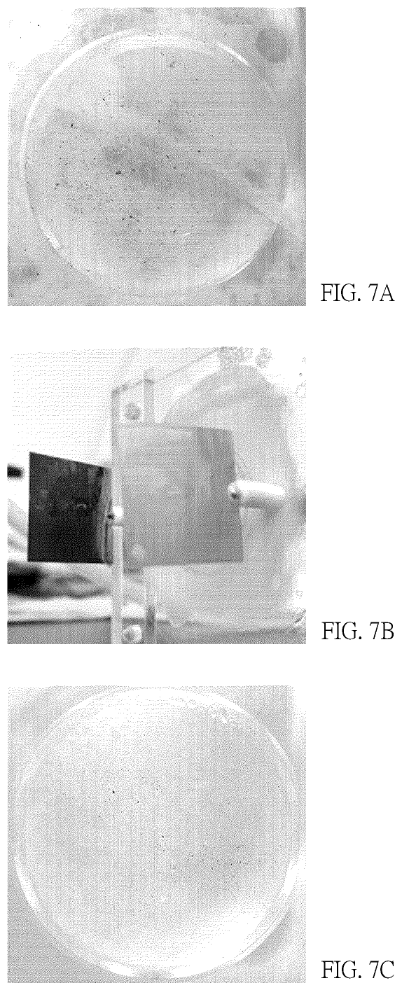

On the other hand, FIGS. 7A, 7B, and 7C illustrate the deterioration of the conventional electrode and the precipitates in the chamber. Referring to FIG. 7A, many precipitates and/or impurities are generated in the chamber of the platinum electrode set. During the process of transparentizing the biological tissues, the generation of the precipitates and/or impurities will cause contamination of the biological tissues. The precipitates and/or impurities not only reduce the transparency effect, but also require time for post-processing the biological tissues to remove or reduce the precipitates and/or impurities. The post-processing will increase the time costs and/or waste of consumables or reagents. In addition, as shown in FIG. 7B, the platinum electrode set undergoes significant deterioration after the transparentizing process. The deteriorated electrode set accelerates the generation of the precipitates and/or impurities. Referring to FIG. 7C, compared to the platinum electrode set, the electrode set of the present invention can effectively reduce the generation of the precipitates and/or impurities in the chamber 120 during the process of transparentizing. Therefore, the electrode set of the present invention can effectively increase the efficiency of transparency and reduce interference with and influence on the biological sample.

By using the first electrode with the carrier metal covered by the coating metal, the coating metal makes the first electrode stable and does not react with the biological sample or the buffer solution. Therefore, the first electrode has a sufficient material stability. In the process of transparentizing, the deterioration of the first electrode, which is used as the anode, will be significantly reduced. Moreover, due to the stability of the electrode set, a DC voltage, constant electrical voltage, and/or constant current can be used to improve the efficiency of electrophoresis without causing electrode deterioration.

The foregoing disclosure is merely preferred embodiments of the present invention and is not intended to limit the claims of the present invention. Any equivalent technical variation of the description and drawings of the present invention of the present shall be within the scope of the claims of the present invention.

What is claimed is:

1. An electrode set for making a biological tissue transparent, comprising:
a first electrode; and
a second electrode,
wherein the first electrode and the second electrode are configured to provide an electrical field to the biological tissue;
wherein the first electrode incudes a carrier metal and a coating metal partially coated on the carrier metal.

2. The electrode set of claim 1, wherein the carrier metal is titanium, and the coating metal is iridium.

3. The electrode set of claim 1, wherein the carrier metal forms a mesh structure.

4. The electrode set of claim 1, wherein the coating metal is a metal oxide.

5. The electrode set of claim 1, wherein the first electrode is a dimensionally stable anode.

6. The electrode set of claim 1, wherein a material of the second electrode is selected from a conductor having a working voltage lower than the first electrode.

7. The electrode set of claim 1, wherein a material of the second electrode is nickel.

8. A device for making a biological tissue transparent, comprising:
an electrode set including:
a first electrode; and
a second electrode,
wherein the first electrode and the second electrode are configured to provide an electrical field to the biological tissue;
wherein the first electrode incudes a carrier metal and a coating metal partially coated on the carrier metal;
a chamber for accommodating the biological tissue, wherein the electrical field is provided in the chamber; and
a power supply configured to provide a working current to the electrode set to generate the electrical field.

9. The device of claim 8, wherein when the biological tissue is transparentized, a working voltage between the first electrode and the second electrode is in a range of 50 V to 100 V.

10. The device of claim 8, wherein the working current is a constant current.

11. The device of claim 8, wherein the working current is generated by providing a constant voltage to the electrode set.

12. The device of claim 8, wherein the carrier metal is titanium, and the coating metal is iridium.

13. The device of claim 8, wherein the carrier metal forms a mesh structure.

14. The device of claim 8, wherein the coating metal is a metal oxide.

15. The device of claim 8, wherein the first electrode is a dimensionally stable anode.

16. The device of claim 8, wherein a material of the second electrode is selected from a conductor having a working voltage lower than the first electrode.

17. The device of claim 8, wherein a material of the second electrode is nickel.
